(19) United States
(12) Patent Application Publication
(10) Pub. No.: US 2016/0242721 A1
(43) Pub. Date: Aug. 25, 2016

(54) APPARATUS AND METHOD FOR FAST ITERATIVE RECONSTRUCTION IN COMPUTED TOMOGRAPHY

(71) Applicants: KABUSHIKI KAISHA TOSHIBA, Tokyo (JP); TOSHIBA MEDICAL SYSTEMS CORPORATION, Tochigi (JP)

(72) Inventors: Yu ZOU, Naperville, IL (US); Zhou Yu, Palatine, IL (US); Daxin Shi, Buffalo Grove, IL (US)

(73) Assignees: KABUSHIKI KAISHA TOSHIBA, Tokyo (JP); TOSHIBA MEDICAL SYSTEMS CORPORATION, Tochigi (JP)

(21) Appl. No.: 14/627,696

(22) Filed: Feb. 20, 2015

(51) Int. Cl. A61B 6/00; G06T 11/00; G06T 7/00; A61B 6/03

(52) U.S. Cl. CPC A61B 6/5211; A61B 6/032; G06T 11/003; G06T 7/0081; G06T 7/0012; G06T 2211/40; G06T 2207/10081; G06T 2207/10116

(57) ABSTRACT

A computed tomography (CT) apparatus and a method for reducing computational complexity in reconstructing a region of interest within an image is provided. The CT apparatus includes a processing circuit that obtains scan data from a scan of an object and computes a system matrix for one view angle of an X-ray source. The system matrix maps an image of the object represented on a circular, symmetric grid to the scan data of the object. Further, the processing circuit reconstructs the image iteratively until a predetermined stopping criterion is satisfied using the scan data and the computed system matrix and generates a sinogram of the reconstructed image based on a forward-projection model. The processing circuit analytically reconstructs a region of interest using the generated sinogram and a predetermined reconstruction kernel.

### FIELD

Embodiments described herein relate to improving the computational speeds of iterative reconstruction methods in computed-tomography (CT).

### BACKGROUND

Radiographic imaging, in its simplest expression, is an X-ray beam traversing an object and a detector relating the overall attenuation per ray. From this conceptual definition, several steps are required to properly construct an image. Several elements affect how the actual image reconstruction is performed.

Typically, mainstream reconstruction methods use an analytical approach to develop a CT image. Iterative statistical methods for 3D tomographic image reconstruction have also gained wide popularity because, in an iterative reconstruction framework, one can model the optics of the imaging system and the statistics of measurements, and further incorporate physical constraints such as object support, non-negativity, object sparsity, piecewise smoothness, motion models, etc. However, such models are difficult to incorporate in a purely analytical reconstruction framework.

Although iterative reconstruction (IR) methods for image reconstruction offer the potential of improved image quality and reduced X-ray dosage, as compared to conventional filtered back-projection methods, the primary bottleneck in the routine use of IR methods is the requirement to solve a complex high dimensional optimization problem. The primary computational bottleneck in IR methods is the forward-projection and back-projection operations, each of which is computationally intensive. The forward and back projectors are linear coefficients that map millions of image pixels onto millions of measurements and vice versa. Thus, the forward-projection and back-projection operations pose a storage issue, and are usually computed on the fly. The repetitive computation of the projection coefficients incurs a high computational cost.

IR techniques also face computation challenges while addressing the problem of a region of interest (ROI) reconstruction. Typically, in an IR technique, an objective function includes all the pixels on an X-ray path in order to ensure that the reconstruction is consistent with the measured data. In such a scenario, in order to obtain a high resolution image of a relatively small ROI, a brute force technique is generally implemented. In the brute force method, the full field of view of the scanner is reconstructed at the same high level of resolution as the small ROI. Thus, the computational cost encountered in the brute force method is high, as the number of pixels that are to be reconstructed is large.

Multi-resolution techniques are an alternative to the brute force method of ROI reconstruction. However in such methods, the full field of view is first reconstructed at a low resolution and then the ROI is reconstructed at a desired high resolution. Although multi-resolution techniques tend to be faster than the brute force method, they require multiple passes of reconstruction and are thus not very efficient, especially when the ROI is small.

Furthermore, in typical IR techniques, a polar-grid image can potentially be employed to accelerate computational speed. However, the regularization technique used in such polar grid representations is a fixed pixel neighborhood, regardless of the pixel size. Therefore, polar grid reconstruction methods are not able to achieve the same image quality as conventional reconstruction methods that use a rectangular grid representation. Accordingly, IR techniques that address the above mentioned problems are desired so that the speed of image reconstruction can be improved.

### BRIEF DESCRIPTION OF THE DRAWINGS

The disclosure will be better understood from reading the description which follows and from examining the accompanying figures. These figures are provided solely as non-limiting examples of the embodiments. In the drawings:

FIG. 1 illustrates an implementation of a computed tomography (CT) system according to one embodiment;

FIG. 2 illustrates an exemplary circular grid for image representation;

FIG. 3 illustrates a flowchart of a process according to one embodiment; and

FIG. 4 illustrates a computer system upon which features of a CT apparatus may be implemented.

### DETAILED DESCRIPTION

According to one embodiment of the present disclosure is provided a computed-tomography (CT) apparatus, comprising a CT scanner including a rotating X-ray source and a detector array configured to receive X-rays emitted from the X-ray source. The CT apparatus also includes a processing circuit that is configured to obtain scan data from a scan of an object and compute a system matrix for one view angle of an X-ray source. The system matrix maps an image of the object represented on a circular, symmetric grid to the scan data of the object. The processing circuit further reconstructs the image iteratively for a predetermined number of iterations using the scan data and the computed system matrix and then generates a sinogram of the reconstructed image based on a forward-projection model. The processing circuit further reconstructs a region of interest using the generated sinogram and a reconstruction algorithm with predetermined reconstruction kernel.

According to one embodiment is provided a method performed by a CT apparatus for reducing computational complexity in reconstructing a region of interest within an image. The method includes obtaining scan data from a scan of an object and computing a system matrix for one view angle of an X-ray source. The system matrix maps an image of the object represented on a circular, symmetric grid to the scan data of the object. The method further includes reconstructing the image iteratively for a predetermined number of iterations using the scan data and the computed system matrix and generating a sinogram of the reconstructed image based on a forward-projection model. The method further reconstructs a region of interest using the generated sinogram and a predetermined reconstruction kernel.

According to one embodiment of the present disclosure is provided a non-transitory computer-readable medium having stored thereon a program that when executed by a computer causes the computer to execute a method. The method includes obtaining scan data from a scan of an object and computing a system matrix for one view angle of an X-ray source. The system matrix maps an image of the object represented on a circular, symmetric grid to the scan data of the object. The method further includes reconstructing the image iteratively for a predetermined number of iterations using the scan data and the computed system matrix and generating a sinogram of the reconstructed image based on a forward-projection model. The method further reconstructs a region of interest using the generated sinogram and a predetermined reconstruction kernel.

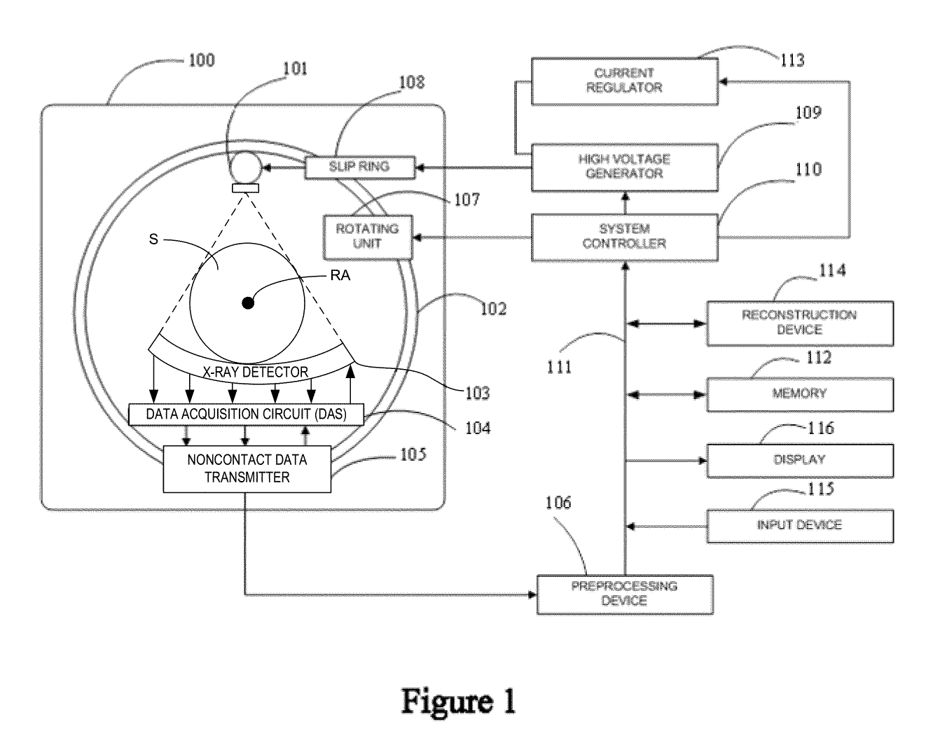

FIG. 1 illustrates an implementation of the radiography gantry included in a CT apparatus or scanner. As shown in FIG. 1, a radiography gantry 100 is illustrated from a side view and further includes an X-ray tube 101, an annular frame 102, and a multi-row or two-dimensional-array-type X-ray detector 103. The X-ray tube 101 and X-ray detector 103 are diametrically mounted across a subject S on the annular frame 102, which is rotatably supported around a rotation axis RA. A rotating unit 107 rotates the annular frame 102 at a high speed, such as 0.4 sec/rotation, while the subject S is being moved along the axis RA into or out of the illustrated page.

According to one embodiment, the X-ray computed tomography apparatus of the present disclosure is described below with reference to the views of the accompanying drawings. Note that the X-ray computed tomography apparatus can include a rotating/rotating-type mechanism in which a X-ray tube and a X-ray detector rotate together around a subject to be examined. Further, according to one embodiment, the CT apparatus can include a stationary/rotating-type mechanism in which a plurality of detectors are arrayed in the form of a ring or plane, and only an X-ray tube rotates around a subject to be examined. It must be appreciated that the features of the present disclosure described herein are applicable to both types of CT apparatuses.

The multi-slice X-ray CT apparatus further includes a high voltage generator 109 that generates a tube voltage applied to the X-ray tube 101 through a slip ring 108 so that the X-ray tube 101 generates X-rays. The X-rays are emitted towards the subject S, whose cross sectional area is represented by a circle. The X-ray detector 103 is located at an opposite side from the X-ray tube 101 across the subject S for detecting the emitted X-rays that have transmitted through the subject S. The X-ray detector 103 further includes individual detector elements or units.

The CT apparatus further includes other devices for processing the detected signals from X-ray detector 103. A data acquisition circuit or a Data Acquisition System (DAS) 104 converts a signal output from the X-ray detector 103 for each channel into a voltage signal, amplifies the signal, and further converts the signal into a digital signal. The X-ray detector 103 and the DAS 104 are configured to handle a predetermined total number of projections per rotation (TPPR). Examples of TPPRs include, but are not limited to 900 TPPR, 900-1800 TPPR, and 900-3600 TPPR.

The above-described data is sent to a preprocessing device 106, which is housed in a console outside the radiography gantry 100 through a non-contact data transmitter 105. The preprocessing device 106 performs certain corrections, such as sensitivity correction on the raw data. A memory 112 stores the resultant data, which is also called projection data at a stage immediately before reconstruction processing. The memory 112 is connected to a system controller 110 through a data/control bus 111, together with a reconstruction device 114, input device 115, and display 116. The system controller 110 controls a current regulator 113 that limits the current to a level sufficient for driving the CT system.

The detectors are rotated and/or fixed with respect to the patient among various generations of the CT scanner systems. The above-described CT system is an example of a combined third-generation geometry and fourth-generation geometry system. In the third-generation system, the X-ray tube 101 and the X-ray detector 103 are diametrically mounted on the annular frame 102 and are rotated around the subject S as the annular frame 102 is rotated about the rotation axis RA. In the fourth-generation geometry system, the detectors are fixedly placed around the patient and an X-ray tube rotates around the patient. In an alternative embodiment, the radiography gantry 100 has multiple detectors arranged on the annular frame 102, which is supported by a C-arm and a stand.

In what follows, a detailed description of the embodiments used to reduce the computational complexity of IR methods in CT is provided. The embodiments described herein are applicable to a fourth generation CT system, a third generation CT system and/or a combination of third and fourth generation CT systems. Specifically, the embodiments described herein are also applicable to a CT system that does not include any photon-counting-detectors.

IR methods typically solve a reconstruction problem by formulating mathematical models of the physics and statistics of the imaging process and the image itself. An important aspect of the modeling process is to find a discrete model of the image and the physical processes, which are continuous in nature. For instance, in a p-dimensional reconstruction problem, the image object can be modeled as a continuous function ƒ(r): [symbol] ^{ρ}→ [symbol] , where r is a vector representing spatial location.

The input to a reconstruction problem is a set of discrete measurements denoted by a vector y. The output of the reconstruction is a discrete image array denoted by a vector x. To define a discrete representation of the image, one can define x to be the samples of ƒ, i.e., xᵢ=ƒ(rᵢ), wherein i is the pixel index and rᵢ are typically chosen to fall on a periodic grid.

The imaging process can be modeled as a mapping from f to y, that is, y=F(f). For example, in 2D parallel-beam CT reconstruction, F is a Radon transform. Once a discrete representation of f is defined, a discrete forward model can be derived as y≈F′(x), mapping from x to y. Upon building the model, a cost function can be formulated that finds the solution that best fits the model. For example, the image can be reconstructed by minimizing a cost function such as:

\hat{c}=argmin{G(F′(x),y)+U(x)}, (1)

wherein G(F′(x),y) is the data mismatch term that penalizes the differences between the a forward projection of the image x and the measurement y according to the forward model F′, and U(x) is the regularization function that penalizes for the roughness in the image.

According to one embodiment of the present disclosure, in order to improve the computational speed of IR methods, a circular image grid is used for image representation. In CT, the X-ray passes are circularly symmetric about the iso-center. In contrast to the widely used rectangular grid for image representation (that is not circularly symmetric), by using a circular grid for image representation it is possible to pre-compute and store a system matrix (i.e., projection operator matrix) for only one view angle, and use the system matrix for successive computations. Thus, a requirement for computing the system matrix during each projection operation for different views is eliminated, thereby obtaining a considerable reduction in the computation complexity. According to one embodiment, the system matrix can be computed for one view angle using a forward model such as a pixel driven method, ray-driven method, Sidon's model, Joseph's method etc.

Note that while employing a polar grid representation, the region near the iso-center of the grid has very small pixel size compared to the region near the periphery of the gird. Thus, using a fixed number of neighborhood pixels will result in a varying neighborhood area, which might result in an under-determined reconstruction problem. Furthermore, due to a variation in the sampling density on the polar grid, the denoise strength can also vary based on the region of the grid.

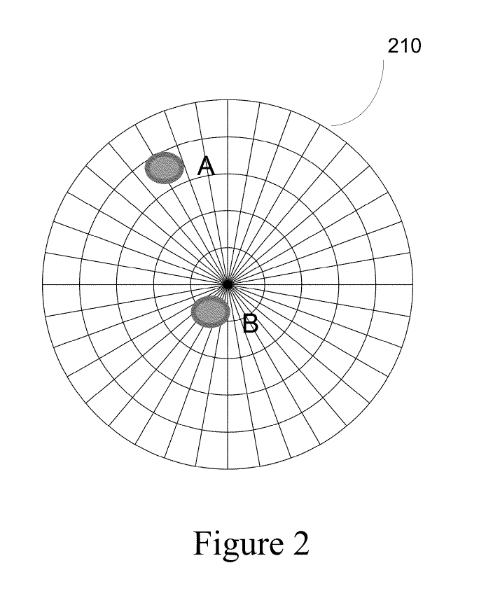

Accordingly, in one embodiment of the present disclosure, and as shown in FIG. 2, the number of neighbors of a pixel is varied based on the location of the pixel within the circular grid. For instance, FIG. 2 depicts a circular grid 210 including two areas represented as A and B. Area B is located near the iso-center of the grid 210 and area A is located near the periphery of the grid 210. In this case, for the areas A and B that have approximately the same area, a pixel in area B (i.e., a pixel located closer to the iso-center) has more neighbors than a pixel in area A (i.e., a pixel lying further away from the iso-center).

Furthermore, in order to ensure that the grid is symmetric over all views and that the denoise strength is uniform over the grid, the number of angular samples taken from the circular grid for image reconstruction is an integer multiple of the number of views. Moreover, the number of image samples taken along the radius of the circular grid can be made to be an integer multiple of the number of detectors in the CT system. Utilizing a sampling scheme as described above provides the flexibility of using any forward projection model, such as a Sidon model, a distance driven model, etc.

According to one embodiment of the present disclosure, the problem of under-determined reconstruction is addressed by utilizing a regularization mechanism, wherein the regularization parameters are properly adjusted to balance noise and resolution. Specifically, in contrast to the commonly used rectangular grid that assumes a fixed regularization parameter for the entire image volume, the regularization parameters for the circular grid are determined based on a change in the sampling rate.

For instance, according to one embodiment, the regularization function U(x) is defined as:

U(x)=Σᵢ₌₀ⁿΣ_{jε∂i}bᵢⱼρ(xᵢ−xⱼ) (2)

where i, j are voxel indices, ρ is the potential function, and ∂i denotes the set of neighbourhood pixels of i. The regularization coefficients bᵢⱼ are adapted based on a change in the sampling size. For example, in the polar grid, the sampling is dense at the iso-center and sparse at the periphery. Thus, the coefficients are varied in grid volume by choosing the parameter bᵢⱼ to be inversely proportional to the squared distance between two pixels. The above regularization function U(x) can be used to minimize a cost function (described with reference to equation (1)) to obtain a solution that best fits the forward model.

In the above-described techniques of reducing the computational complexity of iterative reconstruction methods, the CT system is assumed to perform an axial scan. The system matrix is identical for all view angles as the polar (circular) grid is rotationally symmetric. Thus, computational complexity is reduced by precomputing (and storing) the system matrix and reconstructing the image iteratively by optimizing an objective function using the pre-computed system matrix.

According to one embodiment, the above described mechanisms for reducing the computational complexity of IR methods is also applicable to a CT system performing a helical scan. Note that, in a helical scan, the patient moves along the z-axis, thereby varying the z-component of the system matrix for each view. According to one embodiment, a separable forward model is used while implementing a helical scan. For example, if A(x, y, z, j) denotes the forward model coefficient from a pixel situated at position (x, y, z) to the j^{th} detector element, the separable model satisfies the following condition:

A(x,y,z,j)=A1(x,y,j)*A2(z,j) (3)

where A1 and A2 denote the x-y component and the z-component of the forward model, respectively. In such a case, computational complexity is reduced as A1 remains symmetric for all view angles and A2 is either computed on the fly (only in the z-direction) or pre-computed and stored for each location of the patient on the z-axis.

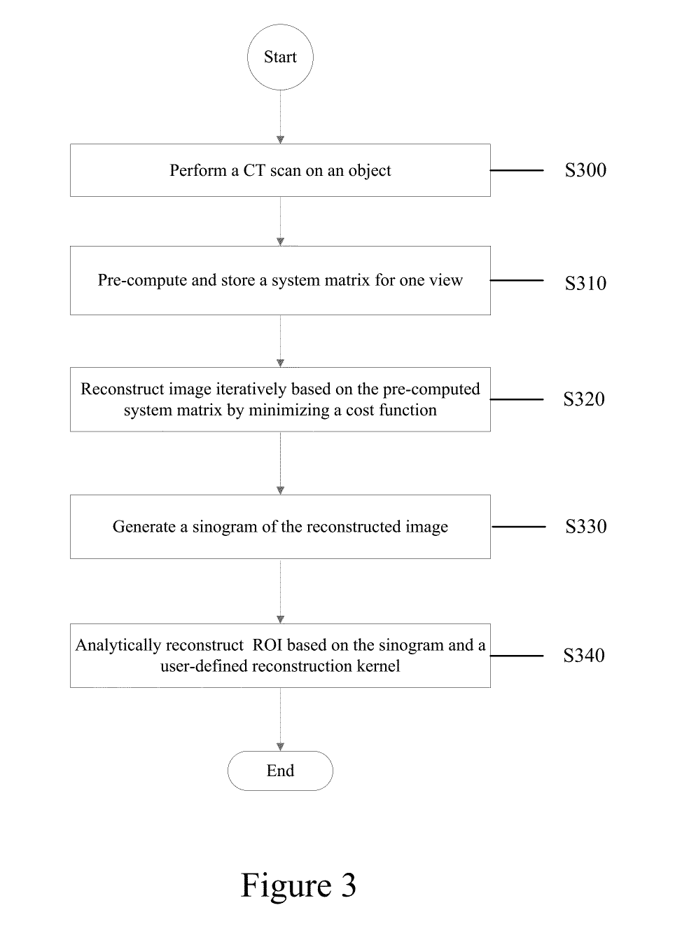

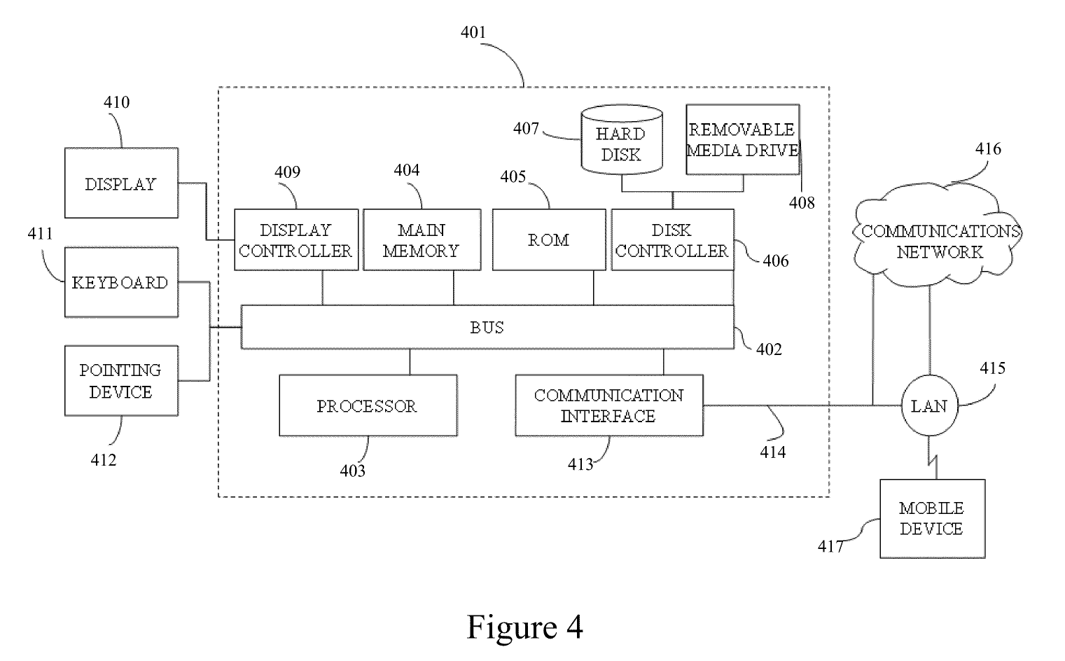

FIG. 3 illustrates a flowchart of a process performed by a CT apparatus/system, according to one embodiment. The apparatus/system has a similar configuration as the computer system 401 illustrated in FIG. 4. Specifically, FIG. 3 illustrates, according to one embodiment, a flowchart depicting the method steps performed by a CT apparatus to reconstruct an image of a region of interest. The ROI reconstruction problem is based on a hybrid reconstruction scheme that eliminates the necessity of performing a multi-pass reconstruction.

In step S300, the CT apparatus performs a scan of an object. Upon obtaining a scan of the object, in step S310, a system matrix (i.e., the projection matrix) is computed for a view angle and stored in a memory included in the CT apparatus.

In step S320, image reconstruction is performed in an iterative manner based on a forward projection and a back projection model. The iterative reconstruction mechanism used herein can be a statistical image reconstruction technique that can be formulated as a maximum likelihood (ML) estimation or maximum a-posteriori (MAP) estimation, numerically solved by conjugate gradient (CG) technique, coordinate descent (CD) mechanism, ordered subsets (OS) method, etc. The iterative reconstruction technique constructs an image such that a cost function (for example equation (1)), which penalizes for the differences between a forward projection of the image and the measurement and furthermore includes a regularization function that penalizes for the roughness in the image, is minimized.

According to one embodiment, the iterative reconstruction is performed until a stopping criterion is satisfied. For instance, the stopping criterion can be a pre-determined number of iterations. Specifically, the iterative reconstruction step is not performed so that full convergence of the final reconstructed image is reached. Rather, the iterative reconstruction step is performed a pre-determined number of times until artifacts caused by noise in the reconstructed image are eliminated. Note that the number of iterations is based on the numerical algorithm that is employed for iterative reconstruction. For instance, the number of pre-determined iterations could be as low as 100 or as high as 1000 iterations.

Further, in step S330, a sinogram of the reconstructed image is generated. A sinogram is a forward projection of the reconstructed image that represents a 2-D array of data containing the projections.

The process then proceeds to step S340, wherein the ROI within an image is reconstructed analytically based on the sinogram data and a user-defined kernel. The reconstruction kernel also referred to as a “filter” or an “algorithm” affects the image quality of the ROI. However, that there is a tradeoff between spatial resolution and noise for each kernel. A smooth kernel generates images with lower noise, but with reduced spatial resolution. A sharp kernel generates images with higher spatial resolution, but increases the image noise. The analytical method could be an FDK algorithm, or more advanced cone beam reconstruction algorithm, such as exact cone beam reconstruction algorithms. Additionally, the method could be further combined with sinogram or image space noise reduction techniques.

The selection of a reconstruction kernel is based on a specific clinical application. For instance, smooth kernels are used in brain exams or liver tumor assessment to reduce image noise and enhance low contrast detectability. The radiation dose associated with such exams is usually higher than that for other exams due to the intrinsic lower contrast between tissues. On the other hand, sharper kernels are used in exams to assess bony structures due to the clinical requirement of better spatial resolution. Lower radiation dose can be used in these exams due to the inherent high contrast of the structures.

Upon reconstructing the ROI analytically based on the user-defined kernel, the process in FIG. 3 terminates. The hybrid reconstruction process as described in FIG. 3 serves two purposes: reconstructing the ROI problem and eliminating the requirement of converting a polar-grid image to a Cartesian-grid image.

Each of the functions of the described embodiments may be implemented by one or more processing circuits. A processing circuit includes a programmed processor (for example, processor 403 in FIG. 4), as a processor includes circuitry. A processing circuit also includes devices such as an application-specific integrated circuit (ASIC) and conventional circuit components arranged to perform the recited functions.

The various features discussed above may be implemented by a computer system (or programmable logic). FIG. 4 illustrates such a computer system 401. The computer system 401 includes a disk controller 406 coupled to the bus 402 to control one or more storage devices for storing information and instructions, such as a magnetic hard disk 407, and a removable media drive 408 (e.g., floppy disk drive, read-only compact disc drive, read/write compact disc drive, compact disc jukebox, tape drive, and removable magneto-optical drive). The storage devices may be added to the computer system 401 using an appropriate device interface (e.g., small computer system interface (SCSI), integrated device electronics (IDE), enhanced-IDE (E-IDE), direct memory access (DMA), or ultra-DMA).

The computer system 401 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system 401 may also include a display controller 409 coupled to the bus 402 to control a display 410, for displaying information to a computer user. The computer system includes input devices, such as a keyboard 411 and a pointing device 412, for interacting with a computer user and providing information to the processor 403. The pointing device 412, for example, may be a mouse, a trackball, a finger for a touch screen sensor, or a pointing stick for communicating direction information and command selections to the processor 403 and for controlling cursor movement on the display 410.

The processor 403 executes one or more sequences of one or more instructions contained in a memory, such as the main memory 404. Such instructions may be read into the main memory 404 from another computer readable medium, such as a hard disk 407 or a removable media drive 408. One or more processors in a multi-processing arrangement may also be employed to execute the sequences of instructions contained in main memory 404. In alternative embodiments, hard-wired circuitry may be used in place of or in combination with software instructions. Thus, embodiments are not limited to any specific combination of hardware circuitry and software.

As stated above, the computer system 401 includes at least one computer readable medium or memory for holding instructions programmed according to any of the teachings of the present disclosure and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes.

Stored on any one or on a combination of computer readable media, the present disclosure includes software for controlling the computer system 401, for driving a device or devices for implementing the invention, and for enabling the computer system 401 to interact with a human user. Such software may include, but is not limited to, device drivers, operating systems, and applications software. Such computer readable media further includes the computer program product of the present disclosure for performing all or a portion (if processing is distributed) of the processing performed in implementing any portion of the invention.

The computer code devices of the present embodiments may be any interpretable or executable code mechanism, including but not limited to scripts, interpretable programs, dynamic link libraries (DLLs), Java classes, and complete executable programs. Moreover, parts of the processing of the present embodiments may be distributed for better performance, reliability, and/or cost.

The term “computer readable medium” as used herein refers to any non-transitory medium that participates in providing instructions to the processor 403 for execution. A computer readable medium may take many forms, including but not limited to, non-volatile media or volatile media. Non-volatile media includes, for example, optical, magnetic disks, and magneto-optical disks, such as the hard disk 407 or the removable media drive 408. Volatile media includes dynamic memory, such as the main memory 404. Transmission media, on the contrary, includes coaxial cables, copper wire and fiber optics, including the wires that make up the bus 402. Transmission media also may also take the form of acoustic or light waves, such as those generated during radio wave and infrared data communications.

Various forms of computer readable media may be involved in carrying out one or more sequences of one or more instructions to processor 403 for execution. For example, the instructions may initially be carried on a magnetic disk of a remote computer. The remote computer can load the instructions for implementing all or a portion of the present disclosure remotely into a dynamic memory and send the instructions over a telephone line using a modem. A modem local to the computer system 401 may receive the data on the telephone line and place the data on the bus 402. The bus 402 carries the data to the main memory 404, from which the processor 403 retrieves and executes the instructions. The instructions received by the main memory 404 may optionally be stored on storage device 407 or 408 either before or after execution by processor 403.

The computer system 401 also includes a communication interface 413 coupled to the bus 402. The communication interface 413 provides a two-way data communication coupling to a network link 414 that is connected to, for example, a local area network (LAN) 415, or to another communications network 416 such as the Internet. For example, the communication interface 413 may be a network interface card to attach to any packet switched LAN. As another example, the communication interface 413 may be an integrated services digital network (ISDN) card. Wireless links may also be implemented. In any such implementation, the communication interface 413 sends and receives electrical, electromagnetic or optical signals that carry digital data streams representing various types of information.

While certain embodiments have been described, these embodiments have been presented by way of example only, and are not intended to limit the scope of the inventions. Indeed, the novel methods described herein may be embodied in a variety of other forms; furthermore, various omissions, substitutions, and changes in the form of the methods and systems described herein may be made without departing from the spirit of the inventions. The accompanying claims and their equivalents are intended to cover such forms or modifications as would fall within the scope and spirit of the inventions.

1. A computed-tomography (CT) apparatus, comprising:
a CT scanner including a rotating X-ray source;
a detector array configured to receive X-rays emitted from the X-ray source; and
a processing circuit configured to
obtain scan data from a scan of an object,
compute a system matrix for one view angle of an X-ray source, the system matrix mapping an image of the object that is represented on a circular, symmetric grid to the scan data of the object,
reconstruct the image iteratively until a predetermined stopping criteria is satisfied using the scan data and the computed system matrix,
generate a sinogram of the reconstructed image based on a forward-projection model, and
reconstruct a region of interest using the generated sinogram and a predetermined reconstruction kernel.

2. The CT apparatus of claim 1, wherein the processing circuit is further configured to sample the image represented on the circular, symmetric grid so that a number of angular samples of the image are equal to an integer multiple of a number of views.

3. The CT apparatus of claim 2, wherein the processing circuit is further configured to sample the image represented on the circular, symmetric grid so that a number of radial samples of the image are equal to an integer multiple of the number of detectors included in the detector array.

4. The CT apparatus of claim 1, wherein the processing circuit is further configured to sample a first portion of the image represented on the circular, symmetric grid that is located near the iso-center of the grid at a higher sampling rate that a second portion of the image that is located near the periphery of the grid.

5. The CT apparatus of claim 4, wherein the processing circuit computes the system matrix for the circular, symmetric grid in which a first image pixel located in the first portion of the image has a higher number of neighbourhood pixels than a second image pixel located in the second portion of the image, an area of the first portion of the image being equal to the area of the second portion of the image.

6. The CT apparatus of claim 1, wherein the processing circuit is further configured to reconstruct the image iteratively by minimizing a cost function, the cost function includes a data mismatch function that corresponds to a first cost incurred for a difference between a forward projection of the reconstructed image and a corresponding measurement, and a regularization function that corresponds to a second cost incurred for roughness in the reconstructed image.

7. The CT apparatus of claim 6, wherein the regularization function includes a plurality of regularization coefficients, each regularization coefficient corresponding to a pair of image pixels, wherein a magnitude of each regularization coefficient is based on the location of the image pixels on the circular, symmetric grid.

8. The CT apparatus of claim 7, wherein the magnitude of each regularization coefficient is inversely proportional to a squared distance between the pair of image pixels.

9. The CT apparatus of claim 1, wherein the forward-projection model is one of a Sidon model and a distance-driven model.

10. A method performed by a CT apparatus for reducing computational complexity in reconstructing a region of interest within an image, the method comprising:
obtaining scan data from a scan of an object;
computing a system matrix for one view angle of an X-ray source, the system matrix mapping an image of the object that is represented on a circular, symmetric grid to the scan data of the object;
reconstructing the image iteratively until a predetermined stopping criteria is satisfied using the scan data and the computed system matrix;
generating a sinogram of the reconstructed image based on a forward-projection model; and
reconstructing a region of interest using the generated sinogram and a predetermined reconstruction kernel.

11. The method of claim 10, further comprising:
sampling the image represented on the circular, symmetric grid so that a number of angular samples of the image are equal to an integer multiple of a number of views.

12. The method of claim 10, further comprising:
sampling the image represented on the circular, symmetric grid so that a number of radial samples of the image are equal to an integer multiple of a number of detectors included in a detector array of the CT apparatus.

13. The method of claim 10, further comprising:
sampling a first portion of the image represented on the circular, symmetric grid that is located near the iso-center of the grid at a higher sampling rate that a second portion of the image that is located near the periphery of the grid.

14. The method of claim 13, wherein the computing step comprises computing the system matrix for the circular, symmetric grid in which a first image pixel located in the first portion of the image has a higher number of neighbourhood pixels than a second image pixel located in the second portion of the image, an area of the first portion of the image being equal to the area of the second portion of the image.

15. The method of claim 10, wherein the reconstructing step comprises reconstructing the image iteratively by minimizing a cost function, the cost function including a data mismatch function that corresponds to a first cost incurred for a difference between a forward-projection of the reconstructed image and a corresponding measurement, and a regularization function that corresponds to a second cost incurred for roughness in the reconstructed image.

16. The method of claim 15, wherein the regularization function includes a plurality of regularization coefficients, each regularization coefficient corresponding to a pair of image pixels, wherein a magnitude of each regularization coefficient is based on the location of the image pixels on the circular, symmetric grid.

17. The method of claim 16, wherein the magnitude of each regularization coefficient is inversely proportional to a squared distance between the pair of image pixels.

18. The method of claim 10, wherein the forward-projection model is one of a Sidon model and a distance-driven model.

19. A non-transitory computer-readable medium having stored thereon a program that when executed by a computer causes the computer to execute a method comprising:
obtaining scan data from a scan of an object;
computing a system matrix for one view angle of an X-ray source, the system matrix mapping an image of the object that is represented on a circular, symmetric grid to the scan data of the object;
reconstructing the image iteratively until a predetermined stopping criteria is satisfied using the scan data and the computed system matrix;
generating a sinogram of the reconstructed image based on a forward-projection model; and
reconstructing a region of interest using the generated sinogram and a predetermined reconstruction kernel.

20. An image processing apparatus, comprising:
a processing circuit configured to
obtain scan data from a scan of an object,
compute a system matrix for one view angle of an X-ray source, the system matrix mapping an image of the object that is represented on a circular, symmetric grid to the scan data of the object,
reconstruct the image iteratively until a predetermined stopping criteria is satisfied using the scan data and the computed system matrix,
generate a sinogram of the reconstructed image based on a forward-projection model, and
reconstruct a region of interest using the generated sinogram and a predetermined reconstruction kernel.
